(19) United States
(12) Patent Application Publication
(10) Pub. No.: US 2025/0243526 A1
(43) Pub. Date: Jul. 31, 2025

(54) PROCESS FOR THE FERMENTATIVE PRODUCTION OF A BIOSURFACTANT

(71) Applicant: Evonik Operations GmbH, Essen (DE)

(72) Inventors: Ezequiel Franco-Lara, Halle (Westf.) (DE); Hans Henning Wenk, Muelheim an der Ruhr (DE); Maciej Olek, Kahl (DE); Tobias Blattert, Bremgarten (CH)

(73) Assignee: Evonik Operations GmbH, Essen (DE)

(21) Appl. No.: 18/854,570

(22) PCT Filed: Apr. 4, 2023
(86) PCT No.: PCT/EP2023/058763

§ 371 Date: Oct. 7, 2024

(30) Foreign Application Priority Data
Apr. 13, 2022 (EP) 22168106.7

(51) Int. Cl. C12P 19/44; C12N 1/14; C12N 1/20; C12R 1/19; C12R 1/385; C12R 1/40

(52) U.S. Cl. CPC C12P 19/44; C12N 1/14; C12N 1/20; C12R 2001/19; C12R 2001/385; C12R 2001/40

(57) ABSTRACT

A process for the fermentative production of a biosurfactant brings a microorganism into contact with a medium that includes a mixture of saccharides. The mixture of saccharides includes glucose and at least one further saccharide which may be fructose, isomaltose, maltose, maltulose, or panose. The microorganism and the medium are provided under conditions where the microorganism is capable of synthesizing the biosurfactant.

### FIELD OF THE INVENTION

The invention relates to a fermentation process for the production of biosurfactants.

### PRIOR ART

As it is reported in literature (Varjani and Upasani, Bioresource Technology 232 (2017): 389-397; Chong and Li, Microb Cell Fact (2017) 16-137), an increasing worldwide production of surfactants is estimated to surpass 24 million tons annually by 2020 (Gudina et al, Biores Technol. 2016; 212:144-50). The longing to replace non-biodegradable petroleum-derived surfactants, which may lead to environmental problems, can be named as main driver of the increase in the biosurfactants demand as environmentally friendly alternatives, especially rhamnolipids (Dobler et al, New Biotechnol. (2016) 33:123-35; Müller et al, J Biotechnol. (2012) 162:366-80; Dusane et al, Biotechnol Genetic Eng Rev. Harding SE editor. (2010) 27:159-184).

As stated by Chong and Li (2017), the most attractive characteristic of biosurfactants is that they are easily biodegradable and cause less toxic impact to the environment, while having similar properties to synthetic surfactants.

Still, the fermentative manufacturing of biosurfactants is quite challenging (Geys et al, Current Opinion in Biotechnology (2014) 30: 66-72). This is given by the nature of the product itself, its raw materials, and the production process. Biosurfactants are highly appreciated in final commercial goods primarily due to their foaming performance, bearing a stable foam during the application of the product, a must-have property. In contrast, excessive foaming is an issue that should be minimized and if possible, avoided, at fermentative production processes (Bator et al, Frontiers in Bioengineering and Biotechnology (2020) 8: Article 899). In this regard, most of the fermentation processes are characterized by increasing product titres, as well as intensive agitation and aeriation of the fermentation broth, which boost the formation of foam in the bioreactor headspace.

This is the major challenge for a commercial successful process, since such stable foam accumulates and increases with forgoing processing time and product titres in the headspace and, if not properly removed or tightly controlled, it can not only cause clogging of pipes, filters and measuring devices, but factually reduces the available reaction volume of the fermenter. Moreover, accumulation of microorganisms usually occurs at the gas-liquid interface of the foam. This results in removal of the biocatalyst from the liquid fermentation broth of the fermenter with the strong consequence of reduced biosurfactant production, as reported for rhamnolipids (Blesken et al., Frontiers in Bioengineering and Biotechnology (2020) 8, Article 572892). Thereby, the use of antifoams during the fermentative process is a common practice, e.g., for rhamnolipids (Beuker et al., AMB Express (2016) 6:124), but hampered due to the large amounts of required antifoam and additional associated costs, as well as increased difficulties in downstream processing (DSP) (Ochsner et al., Adv Biochem Eng Biotechnol (1996) 53: 89-118).

As for the raw materials used in biosurfactant production, mainly glucose, glycerol and several oils have been used as C-source, as detailed reported by Geys et al (Current Opinion in Biotechnology (2014) 30: 66-72), Chong and Li (Microb Cell Fact (2017) 16: 137) and Liu et al (Biotechnology and Bioengineering (2018) 115: 796-814). In the special case of rhamnolipid production with Pseudomonas sp, titres ranging from 0.3 g/l up to 40 g/L are reported when using glucose, from 0.6 g/l up to 30 g/L when using glycerol, and up to remarkable 150 g/I, when using edible vegetable oils like sunflower, corn, coconut, palm, soybean and olive oil.

While plant oils as renewable resources are well suitable substrates for biotensides production, with the additional benefit of acting during fermentative process as antifoams, problems arise in the downstream processing of the fermentation broth, where remaining oils and by-products are difficult and costly to remove to obtain purified final biosurfactants (Heyd et al, Anal Bioanal Chem (2008) 391:1579-1590; Variani et al, Int. J. Innovative Res. Sci. Eng. Technol. 3(2): 9205-9213; Chong and Li Microb Cell Fact (2017) 16:137).

As addressed by Tan and Li (Microb Cell Fact (2018) 17:89), the biggest challenges, low yields, and excessive over-foaming by fermentation-based production, remain when sugars or sugar-containing wastes are used for biosurfactant production. Still, because glucose-based processes are widely established at industrial scale, alternatives to overcome limitations caused mainly by extreme foaming during fermentation have been intensively researched lately, for example by means of recyclable in situ liquid-liquid extraction solvent for foam-free synthesis of rhamnolipids in two-phase fermentation (Demling et al, Green Chem (2020) 22: 8495-8510), or by applying a process-integrated foam fractionation column to separate biosurfactants from medium and bacterial cells (Blesken et al., Frontiers in Bioengineering and Biotechnology (2020) 8, Article 572892). In both cases, glucose was used as main C-source and, although the foam fractioning method was supported by the deletion of genes encoding cell-surface structures (hydrophobic proteins) present on the producing microorganisms P. putida KT2440, none of the process alternatives delivered rhamnolipid titres above 40 g/L under 100 h of operation.

Particularly the extraordinarily increased in foam stability for rhamnolipids produced via fermentation has been reported to be influenced with both, agitation and increase product concentration. The overflowing foam sustained a super high stability in terms of half-time for over 30 min. The major product of rhamnolipid largely contributes to the severe foaming in the fermentation process whereas other products like cells elicit much more limited effects. This explains the severe foaming at late-stage fermentation occurs when rhamnolipid-rich solution is mechanically agitated (Long et al, Journal of Surfactants and Detergents (2016), 19(4): 833-840).

It is an object of the invention to enhance a process for the fermentative production of a biosurfactant in terms of being more convenient to handle regarding over-foaming, fermentation broth viscosities and/or colouring of final product.

### DESCRIPTION OF THE INVENTION

It was found that, surprisingly, by using a well-defined mixture of saccharides, the problem underlying the invention could be solved.

The present invention therefore provides a process for the fermentative production of a biosurfactant as described in claim 1.

One advantage of the present invention is that high titres can be reached.

Another advantage of the present invention is that viscosity of the culture medium is reduced during fermentation

A further advantage is that defoamer consumption is reduced (compared to cultivation with single sugars), i.e. foam formation is reduced

Another advantage is that the reduced viscosity and defoamer consumption lead to lower energy consumption of the mechanical agitation during fermentation

An additional advantage is that final product colour (darkening) is reduced

The instant invention thus provides a process for the fermentative production of a biosurfactant comprising the step of
A) bringing a microorganism into contact with a medium containing a mixture of saccharides consisting of
glucose and at least one further saccharide selected from the group of fructose, isomaltose, maltose, maltulose and panose,
under conditions where the microorganism is capable of synthesizing the biosurfactant.

Within the context of the present invention, “biosurfactants” are understood as meaning all glycolipids produced by fermentation. The term “biosurfactant” also covers glycolipids that are chemically or enzymatically modified after fermentation, as long as structurally a glycolipid remains.

In the context of the present invention, the terms “surfactant” is understood to mean organic substances having interface-active properties that have the ability to reduce the surface tension of water at 20° C. and at a concentration of 0.5% by weight based on the overall composition to below 45 mN/m. Surface tension is determined by the DuNoüy ring method at 20° C.

Where average values are stated hereinbelow, then, unless stated otherwise, these are number-averaged average values.

Unless stated otherwise, percentages are data in percent by weight.

Wherever measurement values are stated hereinbelow, then, unless stated otherwise, these have been determined at a temperature of 25° C. and a pressure of 1013 mbar.

A preferred process according to the instant invention is characterized in, that said mixture of saccharides accounts for at least 85 wt. %, preferably for at least 90 wt. %, even more preferably for at least 95 wt. %, of all saccharides dissolved in the medium.

Other saccharides that may be dissolved in the medium and are not part of said mixture in the process of the instant invention can be one or more selected from the group of lactose, trehalose, maltotriose, raffinose, saccharose.

It is preferred in the instant invention, if the process is characterized in, that said mixture of saccharides accounts for at least 85 wt. %, preferably for at least 90 wt. %, even more preferably for at least 95 wt. %, of all utilizable carbon source present in the medium.

Utilizable carbon that can be present in the medium as described above may be for example other carbohydrates than listed above, in particular sugars, and/or lipophilic carbon sources such as fats, oils, partial glycerides, fatty acids, fatty alcohols, long-chain saturated or unsaturated hydrocarbons.

Preferably, the process according to the instant invention is characterized in, that in said mixture of saccharides
glucose is contained in an amount of
from 85.0 wt. % to 99.5 wt. %, preferably from 90.0 wt. % to 98.0 wt. %, more preferably from 93.0 wt. % to 97.0 wt. %, and
the sum of the further saccharides is contained in an amount of
from 0.5 wt. % to 15.0 wt. %, preferably from 2.0 wt. % to 10.0 wt. %, more preferably from 3.0 wt. % to 7.0 wt. %,
wherein the percentage by weight refer to said mixture of saccharides contained in the medium.

Preferably, the process according to the instant invention is characterized in, that in the mixture of saccharides
fructose is contained in an amount of
from 0.1 wt. % to 10.0 wt. %, preferably from 0.5 wt. % to 8.0 wt. %, more preferably from 1.5 wt. % to 6.0 wt. %,
wherein the percentage by weight refer to said mixture of saccharides contained in the medium.

Preferably, the process according to the instant invention is characterized in, that in the mixture of saccharides
isomaltose is contained in an amount of
from 0.1 wt. % to 4.0 wt. %, preferably from 0.5 wt. % to 3.0 wt. %, more preferably from 1.0 wt. % to 2.0 wt. %,
wherein the percentage by weight refer to said mixture of saccharides contained in the medium.

Preferably, the process according to the instant invention is characterized in, that in the mixture of saccharides
maltose is contained in an amount of
from 0.1 wt. % to 8.0 wt. %, preferably from 1.0 wt. % to 6.0 wt. %, more preferably from 2.0 wt. % to 5.0 wt. %,
wherein the percentage by weight refer to said mixture of saccharides contained in the medium.

Preferably, the process according to the instant invention is characterized in, that in the mixture of saccharides
maltulose is contained in an amount of
from 0.1 wt. % to 2.0 wt. %, preferably from 0.3 wt. % to 1.5 wt. %, more preferably from 0.4 wt. % to 1.0 wt. %,
wherein the percentage by weight refer to said mixture of saccharides contained in the medium.

Preferably, the process according to the instant invention is characterized in, that in the mixture of saccharides
panose is contained in an amount of
from 0.1 wt. % to 3.0 wt. %, preferably from 0.5 wt. % to 2.0 wt. %, more preferably from 0.7 wt. % to 1.5 wt. %,
wherein the percentage by weight refer to said mixture of saccharides contained in the medium.

Preferably the process according to the instant invention is characterized in, that the biosurfactant present in the medium is higher than 50 g/l, preferably from 60 g/l to 200 g/l, more preferably from 80 g/l to 180 g/l, during at least some period of time of the process according to the instant invention.

Preferably, the process according to the instant invention is characterized in, that the biosurfactant is selected from the group of rhamnolipids, sophorolipids and glucolipids.

The term “rhamnolipids” in the context of the present invention preferably is understood to mean particularly compounds of the general formula (I) and salts thereof,

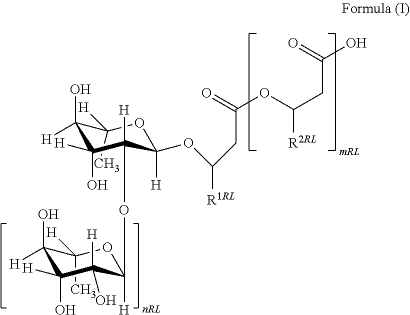

where
mRL=2, 1 or 0,
nRL=1 or 0,
R^{1RL} and R^{2L}=mutually independently, identical or different, organic residues having 2 to 24, preferably 5 to 13 carbon atoms, in particular optionally branched, optionally substituted, particularly hydroxy-substituted, optionally unsaturated, in particular optionally mono-, bi- or tri-unsaturated alkyl residues, preferably those selected from the group consisting of pentenyl, heptenyl, nonenyl, undecenyl and tridecenyl and (CH₂)ₒ—CH₃ where o=1 to 23, preferably 4 to 12.

If nRL=1, the glycosidic bond between the two rhamnose units is preferably in the α-configuration. The optically active carbon atoms of the fatty acids are preferably present as R-enantiomers (e.g. (R)-3-{(R)-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (I) or salts thereof, where nRL=1.

The term “mono-rhamnolipid” in the context of the present invention is understood to mean compounds of the general formula (I) or salts thereof, where nRL=0.

Distinct rhamnolipids are abbreviated according to the following nomenclature:

“diRL-CXCY” are understood to mean di-rhamnolipids of the general formula (I), in which one of the residues R^{1RL} and R^{2L}=(CH₂)ₒ—CH₃ where o=X-4 and the remaining residue R¹ or R²=(CH₂)ₒ—CH₃ where o=Y-4.

“monoRL-CXCY” are understood to mean mono-rhamnolipids of the general formula (I), in which one of the residues R^{1RL} and R^{2L}=(CH₂)ₒ—CH₃ where o=X-4 and the remaining residue R^{1RL} or R^{2RL}=(CH₂)ₒ—CH₃ where o=Y-4.

The nomenclature used therefore does not distinguish between “CXCY” and “CYCX”.

For rhamnolipids where mRL=0, monoRL-CX or diRL-CX is used accordingly.

If one of the abovementioned indices X and/or Y is provided with “:Z”, this signifies that the respective residue R^{1RL} and/or R^{2RL} is equal to an unbranched, unsubstituted hydrocarbon residue having X-3 or Y-3 carbon atoms having Z double bonds.

In the context of the present invention, the term “sophorolipids” preferably is understood as meaning compounds of the general formulae (IIa) and (IIb) and salts thereof

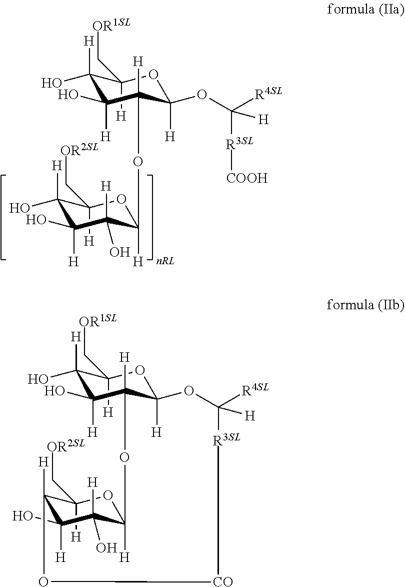

where
R^{1SL}=H or CO—CH₃,
R^{2SL}=H or CO—CH₃,
R^{3SL}=a divalent organic moiety which comprises 6 to 32 carbon atoms and which is unsubstituted or substituted by hydroxyl functions, is unbranched and optionally comprises one to three double or triple bonds,
R^{4SL}=H, CH₃ or a monovalent organic radical which comprises 2 to 10 carbon atoms and which is unsubstituted or substituted by hydroxyl functions, which is unbranched and which optionally comprises one to three double or triple bonds, and
nSL=1 or 0.

Sophorolipids may be produced in accordance with the invention in their acid form or their lactone form.

Preferred processes according to the instant invention produce a sophorolipid in which the ratio by weight of lactone form to acid form is in the range of 20:80 to 80:20, especially preferably in the ranges of 30:70 to 40:60.

To determine the content of sophorolipids in the acid or lactone form in a formulation, refer to EP14111111B, page 8, paragraph [0053].

In connection with the present invention, the term “glucolipids” preferably is understood as meaning compounds of the general formula (III) and salts thereof,

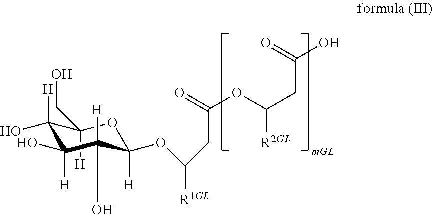

where
mGL=1 or 0,
R^{1GL} and R^{2GL}=independently of one another identical or different organic radical having 2 to 24 carbon atoms, in particular optionally branched, optionally substituted, in particular hydroxy-substituted, optionally unsaturated, in particular optionally mono-, di- or triunsaturated, alkyl radical, preferably one selected from the group consisting of pentenyl, heptenyl, nonenyl, undecenyl and tridecenyl and (CH₂)ₒ—CH₃ where o=1 to 23, preferably 4 to 12.

Distinct glucolipids are abbreviated according to the following nomenclature:

“GL-CXCY” is understood as meaning glucolipids of the general formula (III) in which one of the radicals R^{1GL} and R^{2GL}=(CH₂)ₒ—CH₃ where o=X-4 and the remaining radical R^{1GL} or R^{2GL}=(CH₂)ₒ—CH₃ where o=Y-4.

The nomenclature used thus does not differentiate between “CXCY” and “CYCX”.

If one of the aforementioned indices X and/or Y is provided with “:Z”, then this means that the respective radical R^{1GL} and/or R^{2GL}=an unbranched, unsubstituted hydrocarbon radical with X-3 or Y-3 carbon atoms having Z double bonds.

Preferably the process according to the instant invention is characterized in, that the biosurfactant is selected from rhamnolipids and the microorganism is selected from the group of Pseudomonas putida, Pseudomonas aeruginosa, Serratia rubidaea SNAU02, Escherichia coli and Burkholderia thailandensis.

Preferably the process according to the instant invention is characterized in, that the biosurfactant is selected from rhamnolipids and that the concentration of all rhamnolipids present in the medium is higher than 80 g/l, preferably from 80 g/l to 180 g/l, more preferably from 100 g/l to 160 g/l, during at least some period of time of the process according to the instant invention.

Method—parameters and microorganisms suited for preparing rhamnolipids are disclosed, for example, in EP2786743 and EP2787065.

Fermentation of Pseudomonas, especially Pseudomonas aeruginosa, which are preferably non genetically modified cells, a technology already disclosed in the eighties, as documented e.g. in EP0282942 and DE4127908 can be conducted just as described within the scope of the instant invention by using the special mixture of saccharides. Pseudomonas aeruginosa cells which have been improved for higher rhamnolipid titres by genetical modification can also be used in the context of the instant invention; such cells have for example been disclosed by Lei at al. in Biotechnol Lett. 2020 June; 42(6):997-1002.

Preferably the process according to the instant invention is characterized in, that the biosurfactant is selected from sophorolipids and the microorganism is selected from the group of Starmerella bombicola, Candida bogoiensis, Candida magnoliae, Candida babstae, Candida apicola or Wickerhamiella domericqiae.

If in the process of the instant invention the biosurfactant is selected from sophorolipids and the microorganism is selected from yeast, then mixture of saccharides preferably accounts for at least 25 wt. %, preferably for at least 45 wt. %, even more preferably for at least 70 wt. %, of all utilizable carbon source present in the medium. Other utilizable carbon that is preferably present in the medium in this embodiment of the instant invention are selected from fats, oils, partial glycerides of fatty acids, fatty acids, fatty alcohols and long-chain, preferably C8 to C32, saturated or unsaturated hydrocarbons.

Preferably the process according to the instant invention is characterized in, that the biosurfactant is selected from sophorolipids and that the concentration of all sophorolipids present in the medium is higher than 80 g/l, preferably from 80 g/l to 180 g/l, more preferably from 100 g/l to 160 g/l, during at least some period of time of the process according to the instant invention.

Preferably the process according to the instant invention is characterized in, that the biosurfactant is selected from glucolipids and the microorganism is selected from the group of Pseudomonas putida, Pseudomonas aeruginosa, Escherichia coli, Serrdia rubidaea, preferably strain ATCC 27593, and Burkholderia thailandensis.

Preferably the process according to the instant invention is characterized in, that the biosurfactant is selected from glucolipids and that the concentration of all glucolipids present in the medium is higher than 50 g/l, preferably from 50 g/l to 120 g/l, more preferably from 60 g/l to 100 g/l, during at least some period of time of the process according to the instant invention.

Method—parameters and microorganisms suited for preparing of glucolipids are disclosed, for example in WO2019154970.

Preferably the process according to the instant invention is characterized in, that it comprises the step of B) purifying the biosurfactant, preferably by separating it from the microorganism and/or at least parts of the medium.

The examples adduced hereinafter describe the present invention by way of example, without any intention that the invention, the scope of application of which is apparent from the entirety of the description and the claims, be restricted to the embodiments specified in the examples.

### EXAMPLES

### Example 1: Production of Rhamnolipids with Pseudomonas on Glucose and Fructose

A fermentation is carried out using a Pseudomonas putida strain pBBR1MCS2-Plac-rhIABC-T-Pfac-rhIC-T, the preparation of which is described in US2014296188, comprising the rhamnolipid biosynthesis genes RhIA, RhIB and RhIC. The preculture is carried out in a shaking flask as described in WO2012013554A1. For the main culture, a mineral medium M9 was likewise employed. This medium consists of 2% (w/v) glucose, 0.3% (w/v) KH₂PO₄, 0.679% Na₂HPO₄, 0.05% (w/v) NaCl, 0.2% (w/v) NH₄Cl, 0.049% (w/v) MgSO₄×7 H₂O and 0.1% (v/v) of a trace element solution. This consists of 1.78% (w/v) FeSO₄×7 H₂O, 0.191% (w/v) MnCl₂×7 H₂O, 3.65% (w/v) HCl, 0.187% (w/v) ZnSO₄×7 H₂O, 0.084% (v/v) Na EDTA×2H₂O, 0.03% (v/v) H₃BO₃, 0.025% (w/v) Na₂MoO₄×2 H₂O and 0.47% (w/v) CaCl₂)×2H₂O. The pH of the medium is adjusted to 7.4 using NH₄OH and the medium is subsequently sterilized by means of an autoclave (121° C., 20 min).

The fermentation is conducted in a 2 litre fermenter in a carbon-limited manner via a glucose feed input. The glucose feed input takes place by reference to the dissolved oxygen signal. The dissolved oxygen was regulated at 20% saturation via the stirrer speed. The pH is regulated to 7 via a pH electrode and addition of NH₄OH. To prevent and measure the foaming of the fermentation broth, the defoamer DOW Corning 1500 was added as required. The fermentation was conducted over 4 days.

Furthermore, a medium is prepared and used, where the 2% (w/v) glucose are substituted by 1.98% (w/v) glucose and 0.02% (w/v) fructose, which is called M9*F.

Furthermore, a rhamnolipid producing wildtype strain, P. aeruginosa PAO1, is used.

Relative defoamer amount used

| Strain | M9 | M9*F |
|---|---|---|
| strain pBBR1MCS2-Plac- | 100 | 92 |
| rhlABC-T-Ptac-rhlC-T | | |
| PAO1 | 100 | 93 |

The results show, that the substitution of parts of the glucose by fructose reduces foaming and defoamer consumption, whereas the overall rhamnolipid yield remains the same.

### Example 2: Production of Rhamnolipids with Pseudomonas on Glucose and Maltose

Example 1 is repeated, but a medium is prepared and used, where the 2% (w/v) glucose are substituted by 1.96% (w/v) glucose and 0.04% (w/v) maltose, which is called M9*M. Furthermore, a rhamnolipid producing wildtype strain, P. aeruginosa PAO1, is used.

Relative defoamer amount used

| Strain | M9 | M9*M |
|---|---|---|
| strain pBBR1MCS2-Plac- | 100 | 98 |
| rhlABC-T-Ptac-rhlC-T | | |
| PAO1 | 100 | 99 |

The results show, that the substitution of parts of the glucose by maltose reduces foaming and defoamer consumption, whereas the overall rhamnolipid yield remains the same, but to a lesser extend than substitution by fructose.

After separating off the cells of the fermentations described above by means of centrifugation at 10 000 g, the fermentation broth is adjusted to a pH of 3.1 by adding concentrated H₂SO₄. By centrifugation again at 500 g, a paste-like solid concentrate is obtained with a rhamnolipid fraction of 45% by weight and a viscosity of >10 000 mPas.

The relative colour was determined via the Platinum-Cobalt Scale:

Relative colour

| Strain | M9 | M9*M |
|---|---|---|
| strain pBBR1MCS2-Plac- | 100 | 83 |
| rhlABC-T-Ptac-rhlC-T | | |
| PAO1 | 100 | 89 |

The results show, that the substitution of parts of the glucose by maltose results in a purer rhamnolipid composition.

### Example 3: Production of Rhamnolipids with Pseudomonas on Glucose and Maltose and Fructose

Example 1 is repeated, but a medium is prepared and used, where the 2% (w/v) glucose are substituted by 1.96% (w/v) glucose and 0.03% (w/v) maltose and 0.01% (w/v) fructose, which is called M9*FM.

Furthermore, a rhamnolipid producing wildtype strain, P. aeruginosa PAO1, is used.

Relative defoamer amount used

| Strain | M9 | M9*FM |
|---|---|---|
| strain pBBR1MCS2-Plac- | 100 | 90 |
| rhlABC-T-Ptac-rhlC-T | | |
| PAO1 | 100 | 91 |

The results show, that the substitution of parts of the glucose by maltose and fructose reduces foaming and defoamer consumption in a synergistic way, whereas the overall rhamnolipid yield remains the same.

After separating off the cells of the fermentations described above by means of centrifugation at 10 000 g, the fermentation broth is adjusted to a pH of 3.1 by adding concentrated H₂SO₄. By centrifugation again at 500 g, a paste-like solid concentrate is obtained with a rhamnolipid fraction of 45% by weight and a viscosity of >10 000 mPas.

The relative colour was determined via the Platinum-Cobalt Scale:

Relative colour

| Strain | MF9 | M9*FM |
|---|---|---|
| strain pBBR1MCS2-Plac- | 100 | 85 |
| rhlABC-T-Ptac-rhlC-T | | |
| PAO1 | 100 | 87 |

The results show, that the substitution of parts of the glucose by maltose results in a purer rhamnolipid composition.

### Example 4: Production of Rhamnolipids with Pseudomonas on Glucose and Isomaltose

Example 2 is repeated, but a medium is prepared and used, where the 2% (w/v) glucose are substituted by 1.96% (w/v) glucose and 0.04% isomaltose, which is called M9*l.

The relative colour was determined via the Platinum-Cobalt Scale:

Relative colour

| Strain | M9 | M9*I |
|---|---|---|
| strain pBBR1MCS2-Plac- | 100 | 92 |
| rhlABC-T-Ptac-rhlC-T | | |
| PAO1 | 100 | 95 |

The results show, that the substitution of parts of the glucose by isomaltose results in a purer rhamnolipid composition.

### Example 5: Production of Rhamnolipids with E. coli on Glucose and Panose or Glucose and Maltulose

Production of rhamnolipids is carried out as described in example 10 of EP2598646 in recombinant E. coli M110 pBBR1MCS-2::ABC cells.

CMP medium is used. This consists of 2% (w/v) glucose, 0.007% (w/v) KH₂PO₄, 0.11% Na₂HPO₄×2 H₂O, 0.2% (w/v) NaNO₃, 0.04% (w/v) MgSO₄×H₂O, 0.01% (w/v) CaCl₂)×2H₂O and 0.2% (v/v) of a trace element solution. This consists of 0.2% (w/v) FeSO₄×7 H₂O, 0.15% (w/v) MnSO₄×H₂O and 0.06% (w/v) (NH₄)MO₇O₂₄×4 H₂O. The pH of the medium is adjusted to 6.7 using NaOH.

Additionally medium is prepared and used, where the 2% (w/v) glucose are substituted by 1.98% (w/v) glucose and 0.02% panose, which is called CMP*P. and where the 2% (w/v) glucose are substituted by 1.99% (w/v) glucose and 0.01% maltulose, which is called CMP*M.

The viscosities of the final fermentation broth is measured using a Rheometer (Anton Haak) with concentrical cylinders geometry at a constant shear rate of 100 1/s.

Relative viscosity

| Strain | CMP | CMP*P | CMP*M |
|---|---|---|---|
| E. coli W3110 pBBR1MCS-2::ABC | 100 | 93 | 94 |

The results show, that the substitution of parts of the glucose by panose or maltulose results in lower fermentation broth viscosities. This is an advantage, as less energy needs to be introduced into the vessel during fermentation.

### Example 6: Production of Glycolipids with Pseudomonas on Glucose and Fructose

Production of glucolipids is carried out as described in example 2 of WO2019154970 in recombinant P. putida BS-PP-368 cells in a 1 litre Dasgip fermenter.

Additionally, the pure 500 g/L glucose feed (“G”) is replaced by a 485 g/L glucose plus 15 g/l fructose (G*F).

Relative defoamer amount used

| Strain | G | G*F |
|---|---|---|
| P. putida BS-PP-368 | 100 | 94 |

The results show, that the substitution of parts of the glucose by fructose reduces foaming and defoamer consumption, whereas the overall glucolipid yield remains the same.

### Example 7: Production of Glucolipids with Pseudomonas on Glucose and Maltose

Production of glucolipids is carried out as described in example 2 of WO2019154970 in recombinant P. putida BS-PP-368 cells in a 1 litre Dasgip fermenter.

Additionally, the pure 500 g/L glucose feed (“G”) is replaced by a 490 g/L glucose plus 10 g/l maltose (G*M).

Cells are separated by centrifugation at 10.000 g for 20 minutes. The fermentation broth is separated as the supernatant and adjusted to pH 3.1 by addition of concentrated H₂SO₄.

After a second centrifugation at 5.000 g for 20 minutes the aqueous upper phase is separated off and the remaining lower phase is a concentrate, which had a content of more than 50 wt.-% of glucolipids.

The relative colour is determined via the Platinum-Cobalt Scale:

Relative colour

| Strain | G | G*M |
|---|---|---|
| P. putida BS-PP-368 | 100 | 92 |

The results show, that the substitution of parts of the glucose by maltose results in a purer rhamnolipid composition.

### Example 8: Production of Glucolipids with Pseudomonas on Glucose and Panose or on Glucose and Maltose or Glucose and Panose and Maltose

Production of glucolipids is carried out as described in example 7.

Additionally, the pure 500 g/L glucose feed (“G”) is replaced by a 496 g/L glucose plus 4 g/l panose feed(G*P), by a 496 g/L glucose plus 4 g/l maltose (G*M) feed and by a 496 g/L glucose plus 2 g/l maltose plus 2 g/l panose (G*MP) feed.

The viscosities of the final fermentation broth is measured using a Rheometer with concentrical cylinders geometry at constant a shear rate of 100 1/s.

Relative viscosities

| Strain | G | G*P | G*M | G*MP |
|---|---|---|---|---|
| P. putida BS-PP-368 | 100 | 88 | 86 | 84 |

The results show, that the substitution of parts of the glucose by panose or maltose results in lower fermentation broth viscosities and that panose and maltose exert a synergistic effect. This is helpful, as less energy needs to be introduced into the vessel during fermentation.

### Example 9: Production of Sophorolipids with S. Bombicola on Glucose and Fructose

Sophorolipids are produced as described in example 12 of WO2011061032 in the wildtype strain S. bombicola ATCC 22214 and the recombinant strain S. bombicola ATCC 22214 sbg3-hyg. The medium used for producing the sophorolipids SL is composed of 0.1% KH₂PO₄, 0.5% MgSO₄×7 H₂O, 0.01% FeCl₃, 0.01% NaCl, 0.4% yeast extract, 0.1% urea, 10.5% rapeseed oil and 10% glucose. The pH is brought to 4.5.

Furthermore, a medium is prepared and used, where the 10% (w/v) glucose are substituted by 9.9% (w/v) glucose and 0.1% fructose, which is called SL*F.

The relative foam height in the flasks as well as the sophorolipid concentration is determined

Relative foam height

| Strain | M9 | M9*F |
|---|---|---|
| S. bombicola ATCC 22214 | 100 | 82 |
| S. bombicola ATCC 22214 | 100 | 67 |
| sbg3-hyg | | |

The results show, that the substitution of parts of the glucose by fructose prevents foaming, whereas the overall sophorolipid yield remains the same.

### Example 10: Production of Sophorolipids with S. bombicola on Glucose and Isomaltose or Glucose and Maltose

Sophorolipids are produced according to example 2 of Example 2 WO2021236904 (M).

A second analogous experiment is performed with the glucose being substituted by a 99:1 mixture of glucose and isomaltose (M*I).

A second analogous experiment is performed with the glucose being substituted by a 99:1 mixture of glucose and maltose (M*M).

The relative colour was determined via the Platinum-Cobalt Scale:

Relative colour

| Strain | M | M*I | M*M |
|---|---|---|---|
| S. bombicola ATCC | 100 | 96 | 95 |
| 22214 | | | |

The results show, that the substitution of parts of the glucose by isomaltose or maltose results in a purer sophorolipid composition.

1. A process for the fermentative production of a biosurfactant comprising:
bringing a microorganism into contact with a medium comprising a mixture of saccharides consisting of glucose and at least one further saccharide selected from the group consisting of fructose, isomaltose, maltose, maltulose, and panose, under conditions where the microorganism is capable of synthesizing the biosurfactant.

2. The process according to claim 1, wherein said mixture of saccharides accounts for at least 85 wt. %, of all saccharides dissolved in the medium.

3. The process according to claim 1, wherein said mixture of saccharides accounts for at least 85 wt. % of all utilizable carbon sources present in the medium.

4. The process according to claim 1 wherein said mixture of saccharides comprises glucose in an amount of from 85.0 wt. % to 99.5 wt. %, and
a sum of the further saccharides is contained in an amount of from 15.0 wt. % to 0.5 wt. %,
wherein the percentages by weight refer to said mixture of saccharides contained in the medium.

5. The process according to claim 1, wherein the mixture of saccharides comprises fructose in an amount of from 0.1 wt. % to 10.0 wt. %, and wherein the percentage by weight refers to said mixture of saccharides contained in the medium.

6. The process according to claim 1, wherein the mixture of saccharides comprises isomaltose in an amount of from 0.1 wt. % to 4.0 wt. %, and wherein the percentage by weight refers to said mixture of saccharides contained in the medium.

7. The process according to claim 1, wherein the mixture of saccharides comprises maltose in an amount of from 0.1 wt. % to 8.0 wt. %, and wherein the percentage by weight refers to said mixture of saccharides contained in the medium.

8. The process according to claim 1, wherein the mixture of saccharides comprises maltulose in an amount of from 0.1 wt. % to 2.0 wt. %, and wherein the percentage by weight refers to said mixture of saccharides contained in the medium.

9. The process according to claim 1, wherein the mixture of saccharides comprises panose in an amount of from 0.1 wt. % to 3.0 wt. %, and wherein the percentage by weight refers to said mixture of saccharides contained in the medium.

10. The process according to claim 1, wherein the biosurfactant is selected from the group consisting of rhamnolipids, sophorolipids and glucolipids.

11. The process according to claim 1, wherein the biosurfactant is selected from the group consisting of rhamnolipids and the microorganism is selected from the group consisting of Pseudomonas putida, Pseudomonas aeruginosa, Escherichia coli and Burkholderia thailandensis, or
the biosurfactant is selected from the group consisting of sophorolipids and the microorganism is selected from the group consisting of Starmerella bombicola, Candida bogoriensis, Candida batistae, Candida apicola and Wickerhamiella domericqiae, or
the biosurfactant is selected from the group consisting of glucolipids and the microorganism is selected from the group consisting of Pseudomonas putida, Pseudomonas aeruginosa, Escherichia coli and Burkholderia thailandensis.

12. The process according to claim 2, wherein said mixture of saccharides accounts for at least 95 wt. % of all saccharides dissolved in the medium.

13. The process according to claim 3, wherein said mixture of saccharides accounts for at least 95 wt. % of all utilizable carbon sources present in the medium.

14. The process according to claim 4, wherein said mixture of saccharides comprises glucose in an amount of from 93.0 wt. % to 97.0 wt. %, and the sum of the further saccharides is contained in an amount of from 7.0 wt. % to 3.0 wt. %.

15. The process according to claim 5, wherein the mixture of saccharides comprises fructose in an amount of from 1.5 wt. % to 6.0 wt. %.

16. The process according to claim 6, wherein the mixture of saccharides comprises isomaltose in an amount of from 1.0 wt. % to 2.0 wt. %.

17. The process according to claim 7, wherein the mixture of saccharides comprises maltose in an amount of from 2.0 wt. % to 5.0 wt. %.

18. The process according to claim 8, wherein the mixture of saccharides comprises maltulose in an amount of from 0.4 wt. % to 1.0 wt. %.

19. The process according to claim 9, wherein the mixture of saccharides comprises panose in an amount of from 0.7 wt. % to 1.5 wt. %.
